(19) United States
(12) Patent Application Publication
(10) Pub. No.: US 2022/0062030 A1
(43) Pub. Date: Mar. 3, 2022

(54) POSITIONAL SNORING AND SLEEP APNEA PREVENTION SYSTEM

(71) Applicant: Matthew C. Gertsch, St. George, UT (US)

(72) Inventor: Matthew C. Gertsch, St. George, UT (US)

(21) Appl. No.: 17/410,957

(22) Filed: Aug. 24, 2021

Related U.S. Application Data
(60) Provisional application No. 63/070,920, filed on Aug. 27, 2020.

(51) Int. Cl. A61F 5/56; A61B 5/00

(52) U.S. Cl. CPC A61F 5/566; A61B 5/0051; A61B 5/6803; A61B 2562/0247; A61B 5/0002; A61B 2562/0219; A61B 5/6831

(57) ABSTRACT

A system for reducing or preventing sleep apnea and snoring includes a headband, a sensor (e.g., accelerometer), and a vibrating pad. The sensor may be integrated on a front portion of the headband so as to contact the forehead, while the vibrating pad may be integrated on a rear portion of the headband, contacting the back of the head. The sensor may act as a forehead level, thereby measuring the position of the user's head and sending a signal to the vibrating pad to initiate head repositioning when the head is in an undesirable position.

### CROSS-REFERENCE TO RELATED APPLICATIONS

This application claims the benefit of U.S. Provisional Patent Application Ser. No. 63/070,920 filed on Aug. 27, 2020, which is incorporated herein by reference.

### TECHNICAL FIELD

The present disclosure relates to a device to reduce or prevent sleep apnea and snoring. More particularly, the present disclosure relates to an apparatus that vibrates or inflates to induce a user to reposition their head while sleeping.

### BACKGROUND

Sleep apnea and snoring are two extremely common sleep disturbances. Not only for the individual, but also for their sleep partners. Sleep apnea and snoring both occur when an individual's airway is obstructed when their head and neck are in a straight position. It is in this position that the tongue falls backwards into the airway opening, causing obstruction and cessation of air movement. One of the most effective ways to restore unobstructed airflow is to simply turn the head or neck to the side. This slight, subtle movement is often all that is needed to cause the tongue to become dislodged from the airway, and air is then able to move easier in and out of the lungs. Without these slight head movements to allow air to move more easily, the sleep disturbances may lead to complicated health issues, such as heart problems, high blood pressure, etc.

In the market, there are numerous head bands that are designed to improve the sleep experience of patients. These headbands include technology that study brain activity, monitor head positioning, and have soothing music for meditation and relaxation. For example, there is one sleep product in the market that is inserted into a pillowcase and that self inflates to alter head position when the sound of snoring is detected. However, it does not guarantee a change in head position, nor does it reliably detect head/neck position when snoring is occurring. None of the attempts in the prior art provide sensory feedback for the user to change head positioning.

Accordingly, there is a need for a sleep apnea and snoring device that senses head position and provides a stimulus for a user to change head position. The present disclosure seeks to solve these and other problems.

### SUMMARY OF EXAMPLE EMBODIMENTS

In one embodiment, a system for reducing or preventing sleep apnea and snoring comprises a headband, a sensor (e.g., curvilinear level, accelerometer, gyroscope, etc.), and a vibrating pad (or other stimulus output). The vibrating pad may be removably attachable to the headband on a rear portion. The sensor may be removably attachable on a front portion of the headband so as to contact the forehead. The sensor may act as a forehead level, thereby measuring the position of the user's head and sending a signal to the vibrating pad to initiate head repositioning.

In one embodiment, a system for reducing or preventing sleep apnea and snoring comprises a headband, a positioning sensor, a vibrating pad, and a mobile application. The headband may be a strap, an elastic material, or otherwise capable of circumventing a user's head to secure it in position. Additionally, the sensor may be located in a mandibular advancement device to measure the head/neck position of a user. Further, the sensor may send head position information wirelessly by utilizing a wireless transceiver. In some embodiments, the sensor is a pressure switch that is triggered by the pressure of a user's head.

In one embodiment, a system for reducing or preventing sleep apnea and snoring comprises a vibrating pad and a sensor that may each be removably attachable to an existing headband, such as CPAP straps, or any other type of headwear. In some embodiments, the sensor and vibrating pad are combined into the same accessory.

In one embodiment, a method of preventing sleep apnea comprises using a sensor to determine the position of a user's head. Once the head is determined to be in a straight position, the sensor sends a signal to a vibrating pad, which provides a stimulus to induce head/neck repositioning of the user.

In one embodiment a sleep apnea prevention system may comprise an inflatable component behind the head (either midline or just off midline) that can inflate when a positioning sensor detects an unwanted position, when a pressure sensor detects pressure above a predetermined threshold, or a switch is triggered, thus resulting in a gentle change of head position.

### BRIEF DESCRIPTION OF THE DRAWINGS

FIG. 1A illustrates a top, side perspective view of a system for reducing or preventing sleep apnea and snoring;

FIG. 1B illustrates a block diagram of a positional sensor of a system for reducing or preventing sleep apnea and snoring;

FIG. 1C illustrates a block diagram of a stimulus device of a system for reducing or preventing sleep apnea and snoring;

FIG. 2A illustrates a top, side perspective view of a system for reducing or preventing sleep apnea and snoring comprising a mandibular advancement device;

FIG. 2B illustrates a block diagram of a positional sensor of a system for reducing or preventing sleep apnea and snoring;

FIG. 2C illustrates a block diagram of a stimulus device of a system for reducing or preventing sleep apnea and snoring;

FIG. 3 illustrates a rear perspective view of a system for reducing or preventing sleep apnea and snoring comprising a strap; and

FIG. 4 illustrates a block diagram of a positional sensor of a system for reducing or preventing sleep apnea and snoring comprising a pressure switch;

FIG. 5A illustrates a top, side perspective view of a system for reducing or preventing sleep apnea and snoring comprising an inflatable; and

FIG. 5B illustrates a block diagram of a system for reducing or preventing sleep apnea and snoring comprising an inflatable.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As discussed earlier, there is a need for a device and system that senses or otherwise determines head position and provides a stimulus for a user to change head position. The system for reducing or preventing sleep apnea and snorting disclosed herein seeks to solve these and other problems.

There have been many attempts to curtail the effects of sleep apnea and snoring. However, many of these attempts have shortcomings that lead to an ineffective approach and result. In particular, the sleep apnea and snoring prevention devices on the market often seek to solve the superficial issue, such as preventing snoring, instead of addressing the underlying issue and why it is occurring.

On the other hand, the system for reducing or preventing sleep apnea and snoring described herein seeks to solve the underlying issues of sleep apnea and snoring. Generally, the system for reducing or preventing sleep apnea and snoring comprises a positioning sensor for providing information about the position of the head and neck, and a stimulus, such as a vibrating pad, to induce the user to reposition their head. For example, the positioning sensor would detect when the user/patient's forehead is level with the ceiling, or within a few degrees of that position, which is the most likely position for airway obstruction to occur. The vibrating pad is placed at the back of the head and is activated when the head is in the airway obstructing position. Our brains and bodies turn away quickly from undesired sounds, vibrations, or other stimuli, so as a result of the vibration, the user turns or tilts their head, which allows the airways to be unobstructed. As a result of the new position, the vibration ceases. By placing the stimulus device (e.g., vibrating pad) on the back of the head, when the undesired head position is detected, the head will naturally want to turn away from the signal, thereby creating a habitual repositioning of the head and neck. Further, to minimize sleep disturbance, the degree of vibration intensity could be decreased to a minimum setting that would result in head/neck repositioning without awakening the user. In some embodiments, the vibratory effect changes with time, such as ascending in vibration strength to induce a user to change their head the longer it remains in the undesired position.

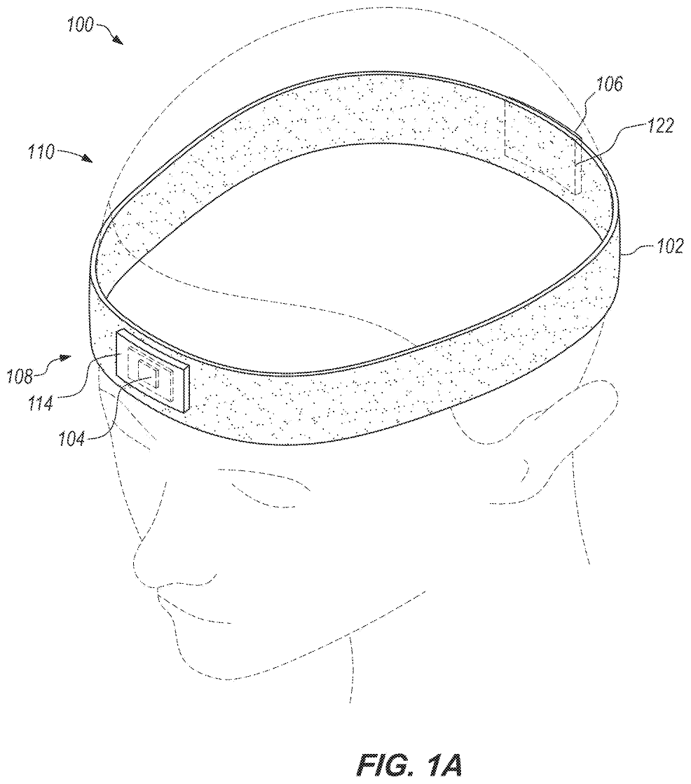

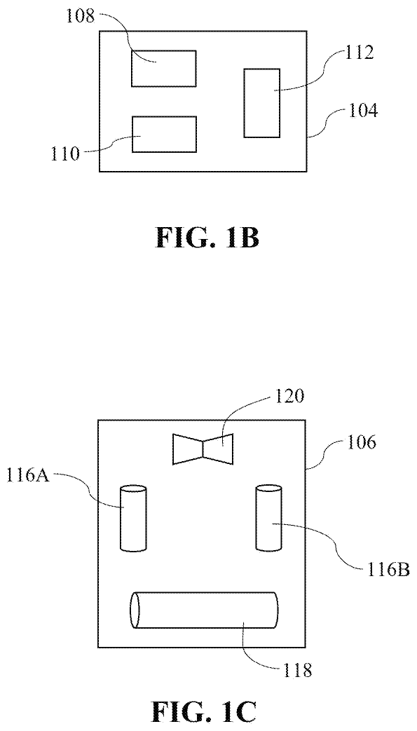

Referring to FIG. 1A, in one embodiment, a system for reducing or preventing sleep apnea and snoring 100 comprises a headband 102, a positioning sensor 104 (e.g., accelerometer, inclinometer, curvilinear level, gyroscope, etc.), and a stimulus device, such as vibrating pad 106. As shown in FIG. 1B, the positioning sensor 104 may comprise a microcontroller 108, a radio frequency transmitter (or transceiver) 110, and one or more sensors for determining relative position, such as an accelerometer 112. The headband 102 may be a strap, elastic material, or otherwise extendable material that may stretch around or circumvent the head of a user and be secured thereon. In some embodiments, the headband 102 may be of a fixed length material. For example, a headband may be custom fitted to a user's head. The positioning sensor 104 may be powered via a battery located in a sensor housing 114 or onboard the sensor positioning sensor 104, or may receive power from a battery located at the vibrating pad 106, with a wire running through the headband from the battery to the positioning sensor 104.

Referring to FIG. 1C, the vibrating pad 106 comprises one or more vibration motors 116A, 116B known in the art so as to generate a vibration felt by a user. It will be appreciated that the same battery 118 may be used to power the vibration motors 116A, 116B of the vibrating pad 106 and the positioning sensor 104 (via wires). Additionally, the battery 118 may be rechargeable using known methods (e.g., USB-C) or may be replaceable. An ON/OFF switch 120 may be used to engage or disengage the battery 118 when not in use.

In use, a user may place the headband 102 on their head so that the vibrating pad 106 is on the rear of the head and may then power on the device using the ON/OFF switch 120, which may be accessible through a housing 122. The microcontroller 108 is pre-programmed to activate the motors 116A, 116B of the vibrating pad 106 when the positioning sensor 104 detects, via the accelerometer 112, that the head is in a substantially upright position (i.e., forehead substantially level/parallel with the floor and ceiling). Once the user tilts their head in response to the vibrations, the accelerometer 112 sends a signal to the microcontroller 108, which then turns off the motors 116A, 116B of the vibrating pad 106. If the user tilts their head back to an upright position, the system repeats and vibration will once again commence. It will be appreciated that the microcontroller 108 may control the motors 116A, 116B via a wireless transceiver 110. However, a wireless transceiver 110 is not required, and the microcontroller 108 may communicate with the motors 116A, 116B using wires that pass through the headband 102.

The positioning sensor 104 may be removably attachable on a front portion 108 of the headband 102 so as to contact the forehead. For example, the positioning sensor 104 may be coupleable to the headband 102 by using hook and loop, magnets, snaps, clips, or any other type of attachment mechanism. The positioning sensor 104 may act as a forehead level, thereby gathering information about the position of the user's head/neck. In some embodiments, the positioning sensor 104 may further comprise a trigger range. Once the head position of the user is determined to be in a straight/upright position (i.e., within the trigger range), the positioning sensor 104 may send a signal to the vibrating pad 106, via the microcontroller or other processor, to initiate head repositioning via the vibrating motors. The positioning sensor 104 and stimulus device 106 may be in electric communication with each other using wires, as noted earlier. However, in one embodiment, the positioning sensor 104 may comprise a wireless transceiver 110 to send and receive signals via a wireless protocol (e.g., Bluetooth®).

After the positioning sensor 104 detects that the head is within the trigger range, it sends a signal to the vibrating pad 106 to commence vibration. The positioning sensor 104 may comprise a microcontroller 108 or other processor capable of processing and transmitting signals. The vibrating pad 106 may be removably attachable to the headband 102 on a rear portion 110. For example, the vibrating pad 106 may comprise hook and look, snaps, clips, magnets, elastic bands, etc. It will be appreciated that the vibrating pad 106 may vary in size, such as height and diameter, to accommodate different users no matter the age or physical characteristics of the user. When the user's head is in a straight position (airway-obstructing position), the vibrating pad 106 begins vibrating to incentivize the user to turn/tilt their head. Because the vibrating pad 106 is located between the user's head and the pillow when vibration begins, the weight of the user's head amplifies the vibration to the user. As a result, the user tilts their head to stop the vibrating. The positioning sensor 104 then detects that the position of the head is in an unobstructed airway position and sends a signal to the vibrating pad 106 to cease vibration.

Further, in some embodiments, the positioning sensor 104 may use an algorithm that only triggers the vibrating pad 106 after the positioning sensor 104 is in the trigger range for a certain time period (e.g., 10 seconds or some other adjustable amount of time). While the positioning sensor 104 and vibrating pad 106 are shown as two separate components on opposite sides of a user's head, such arrangement is not required. For example, the vibrating pad 106 may comprise the positioning sensor 104 so that they are in the same housing and located on the rear of a user's head.

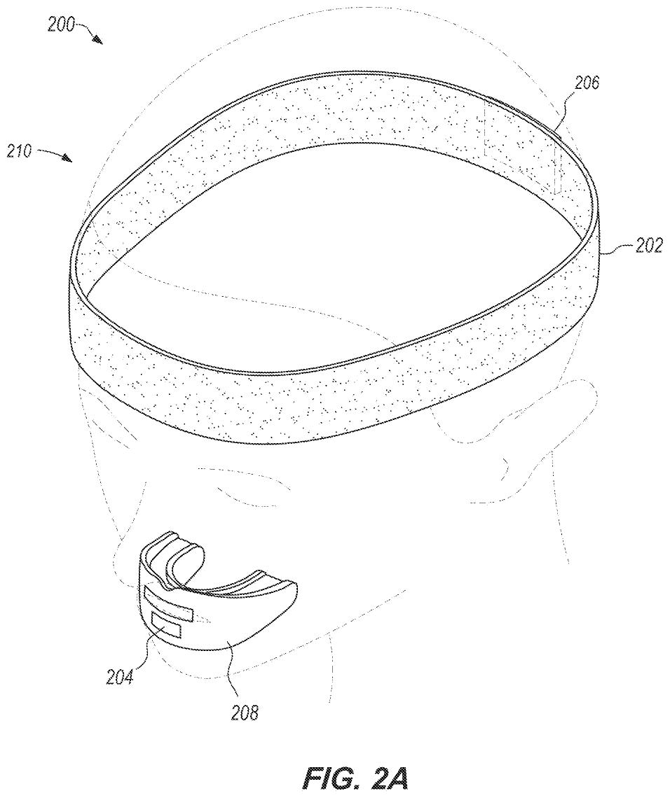

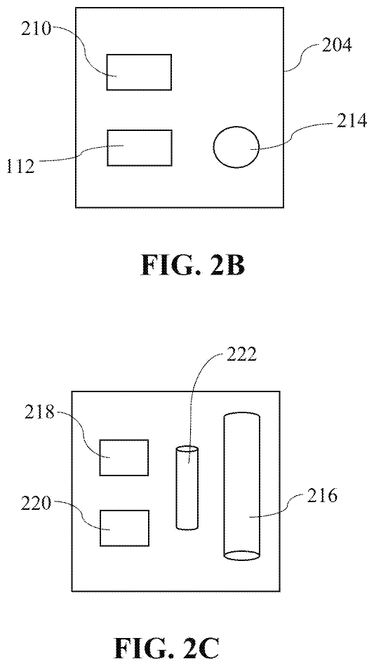

In one embodiment, As shown in FIGS. 2A-2C, a system for reducing or preventing sleep apnea and snoring 200 comprises a headband 202, a positioning sensor 204 (e.g., accelerometer), a vibrating pad 206, and a mobile application. The positioning sensor 204 may be located in a mandibular advancement device 208 to measure the head/neck position of a user. Further, the positioning sensor 204 may send head position information wirelessly by utilizing a wireless transceiver 210. Again, the positioning sensor 204 may comprise a microcontroller 212 or other processor for receiving, processing, and sending signals. It will also be understood that each component, such as the mandibular device 208 and the vibrating pad 206, would each have a battery 214, 216 respectively, which may be rechargeable. Further, the vibrating pad 206 would also have a wireless transceiver 218 and processor 220 for receiving and processing signals from the positioning sensor 204, and one or more vibrating motors 222.

The vibrating pad 206 is ideally positioned at the rear of the head and may be either fixed or removably attachable to the headband 202. With the vibrating pad 206 positioned on the back of the head, the signal is amplified by the weight of the user's head and will encourage the user to turn their heads.

Further, the mobile application may control both the positioning sensor 204 and the vibrating pad 206. The mobile application may connect to the positioning sensor 204 and vibrating pad 206 via Bluetooth® or any other wireless connection. The mobile application may allow the intensity of the vibrating stimulus to be adjusted to reduce sleep disturbance, but still have the effect of head/neck repositioning. The sensitivity of the positioning sensor 204 may also be adjusted to widen or lessen the range of straight-head positioning. In other words, there may be a range where the head and neck of a user may be moved without initiating a head/neck repositioning response, similar to the range described above. The mobile app may further comprise a time component that sets an alarm prior to sending a signal to the vibrating pad 206. In particular, if the head of a user is in an unwanted position for longer than a set period of time (e.g., 10 seconds), then the vibrating pad 206 may be activated. The user may also program other desired options or vectors. It will be appreciated that the vibrating pad 206 trains the brain of a user to slightly reposition the head or neck, thereby creating positive habits and correcting snoring and sleep apnea.

In one embodiment, the mobile application may be programmed to each user, thereby allowing a user to set personal triggering ranges and alerts. Alternatively, a user may set personal alerts directly on the positioning sensor 204, such as by using a dial located on the positioning sensor 204. In either case, personal alerts may address a user's specific needs. For example, some users may only need a slight turn of the head, so for those people, there may be a larger acceptable range of head positioning, and only a small triggering/vibrating range. Further, the mobile application may record and track positioning data (i.e., head positioning) from positioning sensor 204 and the number of alerts issued so that a user may track their body's learning response to the device, among other features. While a mobile application is discussed, devices other than phones may be used, such as any handheld wireless device or a specific input/out device for use only with this system.

In some embodiments, a microphone and/or vibrational sensor may be used in place of a positional sensor. In these embodiments, the microphone may be used to detect the sound of snoring and/or breathing patterns or the vibrational sensor may detect vibrations within the mouth cavity (indicative of snoring), which is then processed by a processor to determine if the sounds or breathing meet or exceed predetermined thresholds. If the thresholds are exceeded (e.g., snoring detected), a signal is sent to the stimulus device to commence stimulation (e.g., vibration, inflation of an inflatable, sound played, etc.).

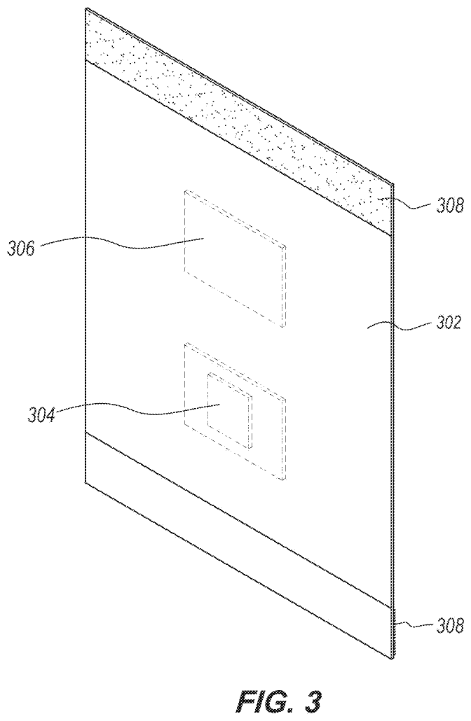

As shown in FIG. 3, in some embodiments, a system for reducing or preventing sleep apnea and snoring 300 comprises a strap 302 comprising a positioning sensor 304 and vibrating pad 306 coupled thereto. The strap 302 may be removably attachable to an existing headband, CPAP straps, or other type of headwear. In particular, the strap 302 may comprise hook and loop couplers 308 so that the strap 302 may be wrapped around an existing headband and secured thereto. However, it will be appreciated that the strap 302 may comprise many other types of securement, such as snaps, magnets, clips, etc. As appreciated, a user may secure the strap 302 to a headband (or other item) and situate it so that the strap 302 is ideally located at the rear of a user's head, allowing the user to feel the vibration when a user's head is upright. While the positioning sensor 304 and vibrating pad 306 are illustrated as separate components, it will be understood that they may be combined into a single component (e.g., single PCB).

In one embodiment, a method of preventing sleep apnea and snoring comprises using a positioning sensor 104, 204, 304 to measure the position of a user's head. Once the head is determined to be in a straight position (potentially obstructed position), the positioning sensor 104, 204, 304 sends a signal to a vibrating pad 106, 206, 306, which provides a stimulus to induce head/neck repositioning of the user.

It will be appreciated that while a vibrating pad 106, 206, 306 has been used as an example stimulus device herein, other types of stimuli may be used to train the brain of a user to reposition the head/neck from a straight position to a slightly turned position. For example, the stimulus device may comprise a heating element, a heat sink and fan to create a cooling effect, a sound-producing speaker, a motor to create a tapping sensation, a motor and pump for inflating objects, a vibrational motor, etc. In other words, as long as the stimulus induces the user to reposition their head, it will be understood that it is within the scope of this invention.

Further, the system for reducing or preventing sleep apnea and snoring may also be used for individuals whose partner prefers that they sleep on one side of the bed. For example, if the user is on the left of the bed, with their sleeping partner on the right side, and their partner is tired of being awakened throughout the night due to frequent turning, the system for reducing or preventing sleep apnea and snoring could be used to vibrate in all undesired positions. Such positions may be programmable by a user using a smartphone, tablet, computer, or other capable device. If snoring is not an issue in this scenario, it could be envisioned that the vibrating pad alerts the user when the head is turned only to the right, with other positions programmed to be acceptable. In contrast, if snoring is a problem, the user that sleeps on the left side of the bed could have the system programmed to vibrate when in any position other than when the head is directed towards the left (away from their sleeping partner).

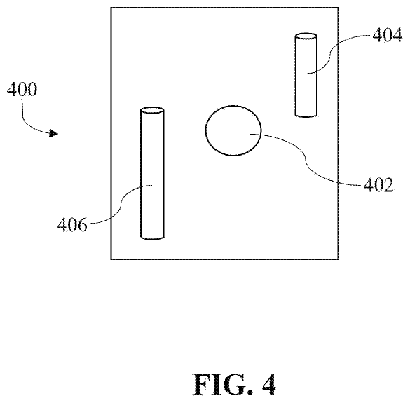

In some embodiments, as shown in FIG. 4, a pressure switch 402 (or sensor) may be used in place of an accelerometer or sensor, eliminating the need for the positioning sensor at the forehead. In other words, a system for reducing or preventing sleep apnea and snoring 400 comprises a pressure switch 402, a vibrating motor 404, and a battery 406. For example, when a user's face is upward, in an airway-obstructing position, the pressure switch 402 on the back of the head is triggered, closing a circuit between the battery 406 and the vibrating motor 404, activating the motor. When the user tilts their head, the switch 402 is released, opening the circuit between the battery 406 and the motor 404. In some embodiments, a pressure sensor may be used that senses the increased pressure, which is processed by a processor which then sends a signal to the vibrating motor 404 to vibrate. Once the user tilts their head and the pressure sensor detects a safe position, it sends a signal to the vibrating motor 404 to cease vibrating.

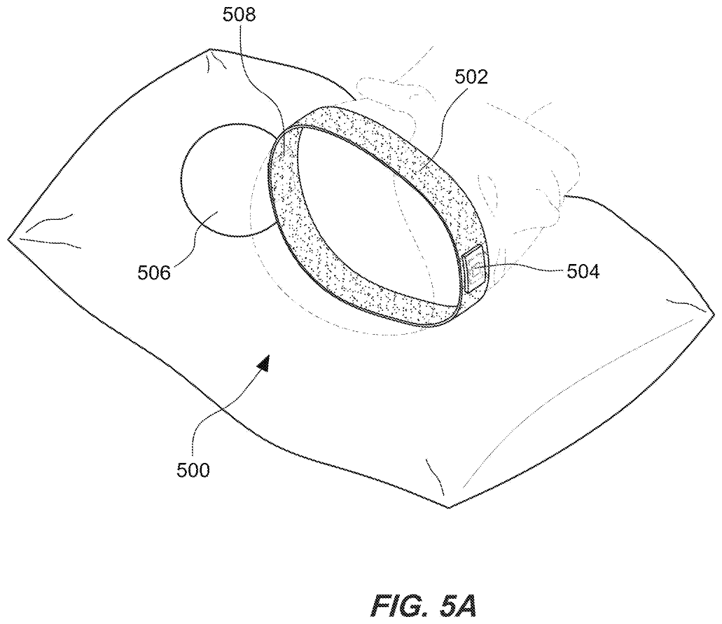

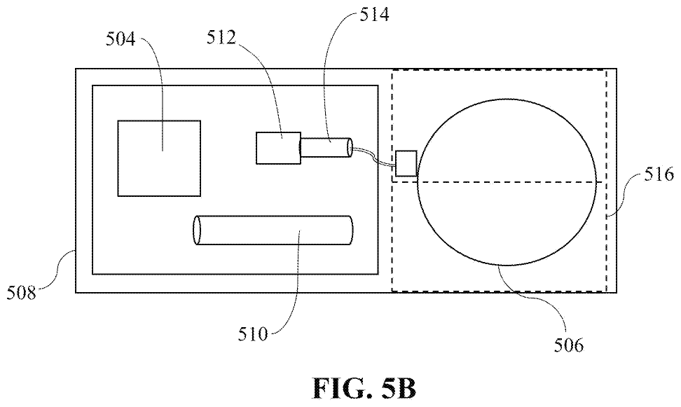

In one embodiment, as shown in FIGS. 5A-5B, a system for reducing or preventing sleep apnea and snoring 500 may comprise a headband 502, a positioning sensor 504, and an inflatable 506. The inflatable 506 is ideally located at the rear of the head (either midline or just off midline) and inflates when the positioning sensor 504 detects an unwanted position, when a pressure sensor detects pressure above a predetermined threshold, or a switch is triggered, thus resulting in a gentle change of head position. In some embodiments, once the head is tilted to the side in response to the inflatable 506, the inflatable 506 may deflate, allowing a user additional range of motion for their head. It will be appreciated that the components may be situated in a housing 508. For example, a battery 510, motor 512, pump 514 coupled to inflatable 506, and positioning sensor 504 may be situated in the housing 508. The inflatable 506 may be of any suitable material, shape, and size. For example, it may be manufactured from rubber, latex, nylon, or other materials capable of reinflation and resistant to popping under pressure. Further, the inflatable 506 may be folded/rolled and stored in the housing 508 behind spring-actuated doors 516, allowing the inflatable (e.g., balloon) to exit as it is inflated.

The system for reducing or preventing sleep apnea and snoring disclosed herein may be integrated into any sleeping headband, mandibular advancement device, and CPAP head strap in the market. It will be appreciated that the system disclosed herein re-trains the brain and body to sleep in more optimal positions for less obstructed and quieter airway movement. After the user's brain and body have been re-trained by the sleep apnea system, the user will be able to sleep without the system and any sleep disruptions.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A system for reducing or preventing sleep apnea and snoring, comprising:
a positioning sensor configured to determine a position of a user's head;
a stimulus device securable to the user's head;
wherein, upon the positioning sensor determining that the user's head is in a first, unwanted position, initiating the stimulus device to induce a user to tilt their head to a second, acceptable position.

2. The system of claim 1, wherein the positioning sensor comprises an accelerometer and a processor.

3. The system of claim 1, wherein the stimulus device comprises at least one vibrating motor.

4. The system of claim 1, wherein the stimulus device comprises an inflatable.

5. The system of claim 1, further comprising a headband.

6. The system of claim 1, further comprising a mandibular advancement device, the positioning sensor located on the mandibular advancement device.

7. The system of claim 6, wherein the mandibular advancement device further comprises a wireless transmitter.

8. The system of claim 1, wherein the positioning sensor and stimulus device are coupled to a strap, the strap comprising hook and loop.

9. The system of claim 1, wherein the positioning sensor comprises a pressure sensor or pressure switch.

10. A system for reducing or preventing sleep apnea and snoring, comprising:
an accelerometer coupled to a microcontroller for determining the position of a user's head;
a vibrating motor actuatable via the microcontroller;
the microcontroller configured to turn on the vibrating motor when the user's head is in a first, upright, position, and to turn off the vibrating motor when the user's head is in a second, tilted, position.

11. The system of claim 11, further comprising a headband.

12. The system of claim 11, further comprising a mandibular advancement device.

13. The system of claim 11, wherein the microcontroller is coupled to a wireless transmitter and the vibrating motor is coupled to a wireless receiver.

14. The system of claim 11, further comprising a strap.

15. A method of using a system for reducing or preventing sleep apnea and snoring, the method comprising:
securing a positional sensor or pressure switch to a user's head;
inducing a user to change the position of their head, using a stimulus device, when the positional sensor or pressure switch is triggered, the stimulus device comprising one or more of:
i. a vibration motor, and
ii. an inflatable.

16. The method of claim 15, wherein the stimulus device is secured to the rear of a user's head.
